(19) United States
(12) Patent Application Publication
(10) Pub. No.: US 2024/0232242 A1
(43) Pub. Date: Jul. 11, 2024

(54) DATA QUALITY CONTROL AND INTEGRATION FOR DIGITAL PATHOLOGY

(71) Applicant: Genentech, Inc., South San Francisco, CA (US)

(72) Inventors: Xingyue GE, Millbrae, CA (US); Phani Sai Kamal LINGAM, Seattle, WA (US)

(21) Appl. No.: 18/611,567

(22) Filed: Mar. 20, 2024

Related U.S. Application Data
(63) Continuation of application No. PCT/US22/44761, filed on Sep. 26, 2022.
(60) Provisional application No. 63/248,354, filed on Sep. 24, 2021.

(51) Int. Cl. G06F 16/34; G06F 16/16; G06T 3/60; G06T 7/11; G06V 20/69; G06V 30/224

(52) U.S. Cl. CPC G06F 16/345; G06F 16/164; G06T 3/60; G06T 7/11; G06V 20/698; G06V 30/224; G06T 2207/20132

(57) ABSTRACT

In one embodiment, a method includes accessing slide files of tissue samples, wherein each slide file is associated with vendor metadata, respectively, generating label metadata, image content metadata, and technical metadata associated with the slide file for each slide file by machine-learning models, performing metadata cross-validation on each slide file based on a comparison of the respective vendor metadata with the respective label metadata, image content metadata, and technical metadata associated with the slide file, generating a report summarizing the slide files based on the metadata cross-validation, wherein the report indicates a number of matches and a number of mismatches from the metadata cross-validation for the slide files, and providing instructions for displaying the report to a user via a user interface, wherein the user interface is operable for the user to view the vendor metadata, label metadata, image content metadata, and technical metadata associated with each slide file.

### CROSS-REFERENCE TO RELATED APPLICATION(S)

This application is a continuation of International Application No. PCT/US2022/044761, filed on Sep. 26, 2022, which claims the benefit of U.S. Provisional Application No. 63/248,354, filed on Sep. 24, 2021, the entire contents of which are incorporated herein by reference in their entirety.

### FIELD OF THE INVENTION

The present disclosure relates to a system and methods for improving data quality control and integration for digital pathology.

### INTRODUCTION

Digital pathology is a sub-field of pathology that focuses on data management based on information generated from digitized specimen slides. Through the use of computer-based technology, digital pathology utilizes virtual microscopy. Glass slides are converted into digital slides that can be viewed, managed, shared and analyzed on a computer monitor. With the practice of whole-slide imaging (WSI), which is another name for virtual microscopy, the field of digital pathology is growing and has applications in diagnostic medicine, with the goal of achieving efficient and cheaper diagnoses, prognosis, and prediction of diseases due to the success in artificial intelligence and machine learning.

### SUMMARY OF PARTICULAR EMBODIMENTS

Herein is provided a system and methods for improving data quality control and integration for digital pathology.

In particular embodiments, a data quality control system may be AI-powered to address the growing needs of data consumption and ensure data quality standards and data integrity across the board. The data quality control system may be particularly suitable for advanced digital pathology. In particular embodiments, the data quality control system may comprise a plurality of independently requestable modules. These modules may comprise a module for automatic label detection and recognition (AutoLDR), a module for classification of hematoxylin and eosin stained slides (CHESS), and a module for tissue identification (TiD). Each module may generate their own metadata, tabular file structures, images, analysis results, reports, and other intermediary files as necessary. In particular embodiments, the data quality control system may comprise a dashboard application built using RShiny framework. The dashboard application may provide the end-user with easy-to-use interface, structured reporting standards, and simple to understand and operate design. In the backend, each module may come with its own scripts running in the background to produce, analyze and validate the slides.

In particular embodiments, a data quality control system may access a plurality of slide files of a plurality of tissue samples, respectively. Each of the plurality of slide files may be associated with vendor metadata, respectively. The data quality control system may then generate, for each of the plurality of slide files by one or more machine-learning models, label metadata, image content metadata, and technical metadata associated with the slide file. In particular embodiments, the data quality control system may perform metadata cross-validation on each of the plurality of slide files based on a comparison of the respective vendor metadata with the respective label metadata, image content metadata, and technical metadata associated with the slide file. The data quality control system may generate a report summarizing the plurality of slide files based on the metadata cross-validation. In particular embodiments, the report may indicate a number of matches and a number of mismatches from the metadata cross-validation for the plurality of slide files. The data quality control system may further provide instructions for displaying, via a user interface, the report to a user. The user interface may be operable for the user to view the vendor metadata, label metadata, image content metadata, and technical metadata associated with each of the plurality of slide files.

### BRIEF DESCRIPTION OF THE DRAWINGS

One or more drawings included herein are in color in accordance with 37 CFR § 1.84. More specifically, FIGS. 3, 4, 10A, 10B, and 11B include one or more high-resolution histopathology images and user interfaces displaying histopathology images, in which color plays a predominant role in enabling one skilled in the art to understand the invention. These color drawings are the only practical medium for disclosing the subject matter to be patented and are necessary to illustrate the invention.

FIG. 1 illustrates an example pipeline flowchart for data integration.

FIG. 2 illustrates an example data flow diagram for data integration.

FIG. 3 illustrates an example thumbnail image.

FIG. 4 illustrates an example assay image.

FIG. 5 illustrates an example process flowchart of the module for automatic label detection and recognition (AutoLDR).

FIG. 6 illustrates an example label image.

FIG. 7A illustrates an example label image.

FIG. 7B illustrates an example processed and oriented label image.

FIG. 7C illustrates example extracted text.

FIG. 8A illustrates an example barcode as a digital encoded label.

FIG. 8B illustrates an example QR code as a digital encoded label.

FIG. 9 illustrates an example empty slide.

FIG. 10A illustrates an example tissue region.

FIG. 10B illustrates an example segmented tissue region.

FIG. 11A illustrates an example dashboard interface with label view.

FIG. 11B illustrates an example user interface with assay view.

FIG. 12 illustrates an example method for data quality control and data integration.

FIG. 13 illustrates an example of a computing system.

### DESCRIPTION

The whole-slide imaging (WSI) may come in various file formats depending on the scanner used. In other words, two or more of the plurality of slide files may be based on different file formats. Accessing and analyzing these different format slides may be difficult at times and require a complex system in place to support them. In particular embodiments, the data quality control system may support a variety of file formats such as the NDPI format (a pathology slide specimen image created by a Hamamatsu slide scanner), the SVS format (a digital slide image file created by an Aperio ScanScope slide scanner), etc. The data quality control system may address the various quality control aspects of the slides from metadata to assessment of slide contents, thereby creating a robust system for doing the advanced checks necessary for high data reliance.

In particular embodiments, the data quality control system may be based on a plurality of independently requestable modules. These modules may comprise a module for automatic label detection and recognition (AutoLDR), a module for classification of hematoxylin and eosin stained slides (CHESS), and a module for tissue identification (TiD). Each module may generate their own metadata, tabular file structures, images, analysis results, reports, and other intermediary files as necessary. In particular embodiments, the data quality control system may comprise a dashboard application built using RShiny framework. The dashboard application may provide the end-user with easy-to-use interface, structured reporting standards, and simple to understand and operate design. In the backend, each module may come with its own scripts running in the background to produce, analyze and validate the slides.

The WSI file size may vary from a few hundreds of megabytes to over multi-gigabytes. Each file may have a pyramid structure with various layers embedded based on the magnification or resolution. In particular embodiments, the data quality control system may deal with the bazel image layer called thumbnail or macro image. In other words, each slide file of the plurality of slide files may comprise a plurality of layers, wherein the plurality of layers may comprise at least a thumbnail image. The data quality control system may use open source tools to read, access and extract information needed from the slides. As an example and not by way of limitation, such tools may comprise OpenSlide. A thumbnail image may have two important regions, one where the assay contents like tissue samples of the slide is present and the other where the label of the slide is present. In other words, the thumbnail image may comprise one or more of an assay content or a label associated with the corresponding slide file. Assay region of the slides may enable us to conduct a wide variety of image analysis techniques to gather insights and understand the slide in a better way. The label region may contain the metadata in various formats. As an example and not by way of limitation, the label may comprise one or more of text or a digital code. Other formats may comprise hand-written text, printed text, or any other digital encoded formats like barcodes, QR codes, etc. This label region may allow us to extract as much structured information as possible and utilize it for data curation, verification of standards and validation of generated metadata.

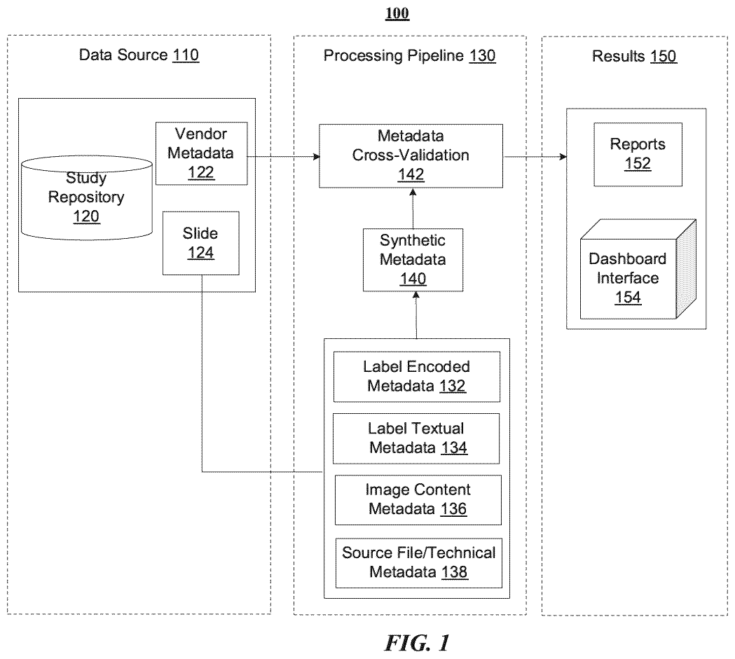

FIG. 1 illustrates an example pipeline 100 flowchart for data integration. Through this pipeline 100, the data quality control system may collect, generate and consume metadata in and from various forms. The current processes in the pipeline may comprise utilizing the slides and metadata structures provided by the vendors from study data repositories, extracting the thumbnail image and generating various types of metadata and aggregating it into a synthetic metadata file, generating reports specific to each module and presenting the use with simplified reports in a dashboard interface for the end-user. As illustrated in FIG. 1, the data quality control system may first access the data source 110. The data source 110 may comprise the study repository 120, which may further comprise vendor metadata 122 and slides 124. After accessing the data source 110, the data quality control system may proceed to the processing pipeline 130. In the processing pipeline 130, the accessed slides 124 may be processed to generate label encoded metadata 132, label textual metadata 134, image content metadata 136, and source file/technical metadata 138. The data quality control system may generate synthetic metadata 140 based on label encoded metadata 132, label textual metadata 134, image content metadata 136, and source file/technical metadata 138. The data quality control system may then perform metadata cross-validation 142 based on the accessed vendor metadata 122 and the synthetic metadata 140. In particular embodiments, the vendors may provide structured metadata (i.e., vendor metadata 122). The vendor metadata 122 may come in with a structure that has a wide variety of data fields beyond the fields in label metadata. As an example and not by way of limitation, information like stain type, anatomical tissue type, or the slide magnification level may be present in the vendor metadata, but not present on the labels. In such cases, the data quality control system may use the image content metadata 136 and source file/technical metadata 138.

As described above, the data quality control system may generate a synthetic metadata 140 file by aggregating the label metadata, image metadata, and technical metadata 138 associated with each of the plurality of slide files 124. Once the data quality control system generates the synthetic metadata 140, the data quality control system may compare all the available common fields from both the vendor metadata 122 and synthetic metadata 140, not just the label metadata. In other words, the comparison of the respective vendor metadata 122 with the respective label metadata may be based on the synthetic metadata 140 file. It may ensure maximum utilization of the extracted metadata. In alternative embodiments, the vendors may not provide structured metadata. In this case, the data quality control system may extract metadata from the label, image content and technical metadata 138 to supply a structured metadata for metadata cross-validation 142. Performing metadata cross-validation 142 may provide one or more technical advantages. A technical advantage may include ensuring data quality standards and data integrity across the board. Another technical advantage may include the ability to determine if it is necessary to communicate with a vendor based on the results of cross-validation, e.g., the results indicating some errors in the original vendor metadata 122. Another technical advantage may include data cleaning (e.g., fixing or removing incorrect, corrupted, incorrectly formatted, duplicate, or incomplete data) for future analysis as clean data for big data analysis of different tasks is essential for accuracy and reliability. After the metadata cross-validation 142, the data quality control system may generate results 150. The results 150 may comprise reports 152 and a dashboard user interface 154.

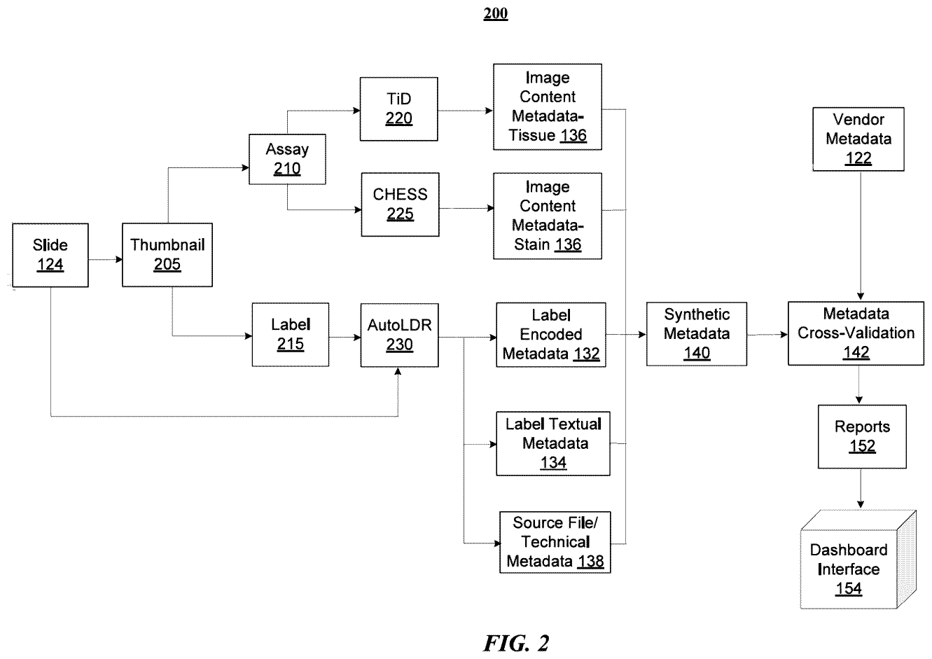

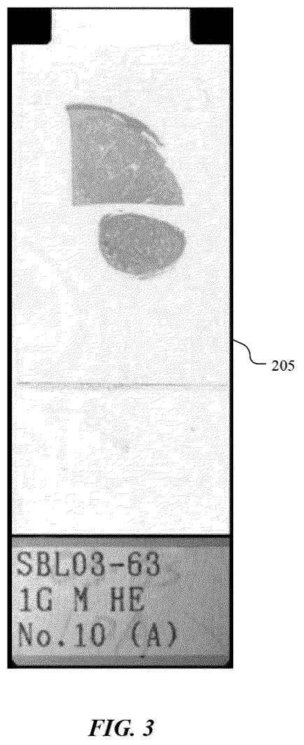

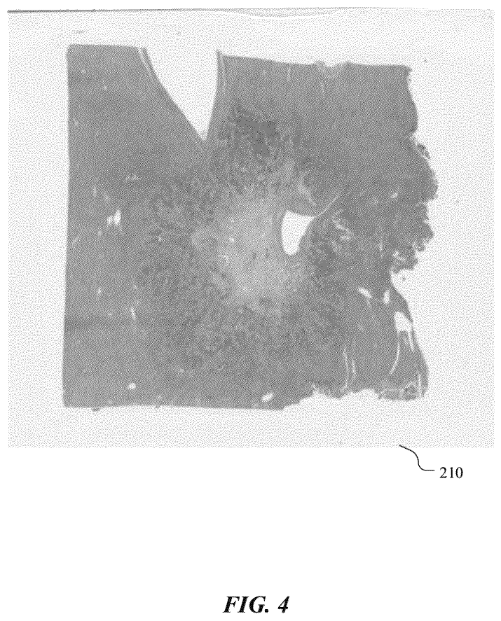

FIG. 2 illustrates an example data flow diagram 200 for data integration. In particular embodiments, the data quality control system may generate thumbnail images 205 from the slides 124. FIG. 3 illustrates an example thumbnail image 205. In particular embodiments, the data quality control system may detect one or more artifacts associated with one or more of the plurality of slide files 124 based on the thumbnail images 205. Correspondingly, the report may further comprise information associated with the detected artifacts. In particular embodiments, the type of artifacts detectable may comprise pre-analytics, staining, and scanning. As an example and not by way of limitation, pre-analytics artifacts may comprise wrong label placement, tissue tears, tissue folding, air bubbles, dust particles, washed-off tissue (missing tissue). As another example and not by way of limitation, staining artifacts may comprise dye precipitation, over/under staining, and edge artifacts. Specifically, the edge artifacts may be explained as follows. During the preparation of the tissue block, the surface layer of the tissue may comprise dark marker/stain. Thus, the residual may be seen when the sections are made from the specific block, resulting in dark edges which may be seen as artifacts that may potentially affect downstream computational processing. As another example and not by way of limitation, scanning artifacts may comprise pen marks, blur rendering, color balance, and stitching error. In particular embodiments, the data quality control system may then generate assay images 210 and labels 215 based on the thumbnail images 205. FIG. 4 illustrates an example assay image 210.

The assay images 210 may be processed by the module for tissue identification (TiD) 220 and the module for classification of hematoxylin and eosin stained slides (CHESS) 225. In particular embodiments, the module for tissue identification (TiD) 220 may generate image content metadata 136 comprising identified tissue. The module for classification of hematoxylin and eosin stained slides (CHESS) 225 may generate image content data 130 comprising classified stain. In particular embodiments, the labels 215 and the slides 124 may be processed by the module for automatic label detection and recognition (AutoLDR) 230. The label metadata may comprise one or more of label encoded metadata 132 or label textual metadata 134. The module for automatic label detection and recognition (AutoLDR) 230 may generate label encoded metadata 132, label textual metadata 134, and source file/technical metadata 138. The data quality control system may generate synthetic metadata 140 based on the image content metadata 136 comprising identified tissue, image content data 130 comprising classified stain, label encoded metadata 132, label textual metadata 134, and source file/technical metadata 138. The data quality control system may then perform metadata cross-validation 142 based on the vendor metadata 122 and the synthetic metadata 140. Generating synthetic metadata 140 and performing metadata cross-validation 142 based on the synthetic metadata 140 and vendor metadata 122 may provide several technical advantages. A technical advantage may include ensuring data quality standards and data integrity as the comparison between the synthetic metadata 140 and the vendor metadata 122 may provide insights of data quality and integrity, which may also help fix the issues, if any. Another technical advantage may include the ability to determine if it is necessary to communicate with a vendor based on the cross-validation. Another technical advantage may include identifying incorrect, corrupted, incorrectly formatted, duplicate, or incomplete data based on cross-validation between the synthetic metadata 140 and vendor metadata 122, which may be further used for data cleaning for future analysis of different tasks. After the metadata cross-validation 142, the data quality control system may generate reports 152 and present the reports 152 via the dashboard user interface 154.

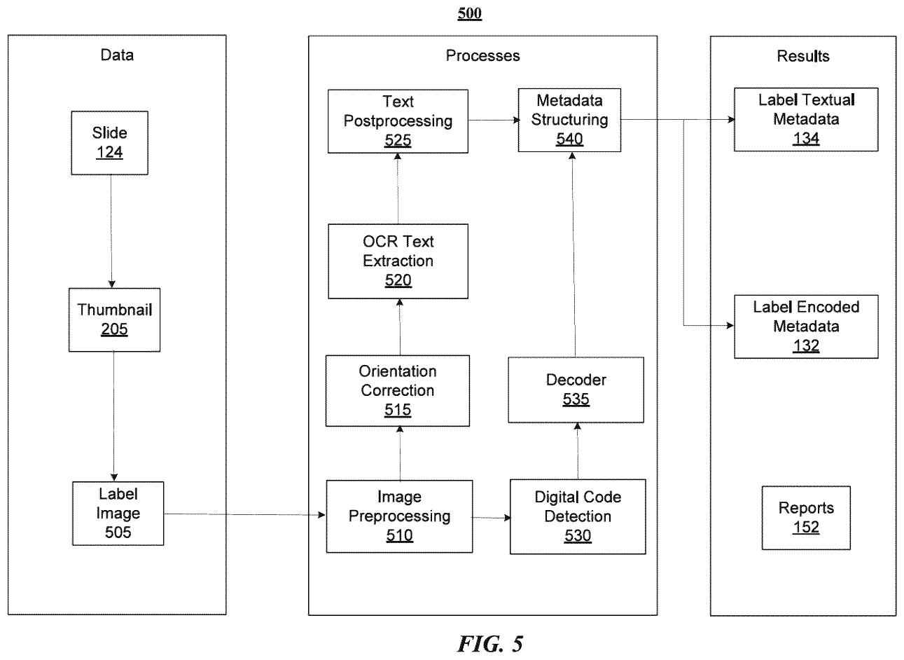

FIG. 5 illustrates an example process flowchart 500 of the module for automatic label detection and recognition (AutoLDR) 230. In the module for automatic label detection and recognition (AutoLDR) 230, the data quality control system may understand the label region of a thumbnail image 205 and extract information. Once the thumbnail image 205 from the file structure of a slide 124 is extracted, the module for automatic label detection and recognition (AutoLDR) 230 may identify the boundaries of the label image 505 within the whole thumbnail image 205 and crop it out for analysis purposes.

When the label metadata comprises label encoded metadata 132, the detailed steps for generating the label encoded metadata 132 may be as follows. For each of the plurality of slide files 124, the module for automatic label detection and recognition (AutoLDR) 230 may extract the thumbnail image 205 of the slide file 124. The thumbnail image 205 may comprise a label 215 associated with the corresponding slide file 124. The module for automatic label detection and recognition (AutoLDR) 230 may then identify boundaries of the label 215 within the thumbnail image 205. The module for automatic label detection and recognition (AutoLDR) 230 may then generate a label image 505 by cropping out the label 215 based on the boundaries of the label 215. The module for automatic label detection and recognition (AutoLDR) 230 may then detect a presence of a digital code in the label image 505. The module for automatic label detection and recognition (AutoLDR) 230 may further generate the label encoded metadata 132 based on decoding the digital code.

When the label metadata comprises label textual metadata 134, the detailed steps for generating the label textual metadata 134 may be as follows. For each of the plurality of slide files 124, the module for automatic label detection and recognition (AutoLDR) 230 may extract the thumbnail image 205 of the slide file 124. The thumbnail image 205 may comprise a label 215 associated with the corresponding slide file 124. The module for automatic label detection and recognition (AutoLDR) 230 may then identify boundaries of the label 215 within the thumbnail image 205. The module for automatic label detection and recognition (AutoLDR) 230 may then generate a label image 505 by cropping out the label 215 based on the boundaries of the label 215. The module for automatic label detection and recognition (AutoLDR) 230 may then preprocess the label image 505. As an example and not by way of limitation, the preprocessing may comprise one or more of image blurring, illumination correction, or thresholding. The module for automatic label detection and recognition (AutoLDR) 230 may then detect text in the preprocessed label image. The module for automatic label detection and recognition (AutoLDR) 230 may further generate the label textual metadata 134 based on optical character recognition on the detected text.

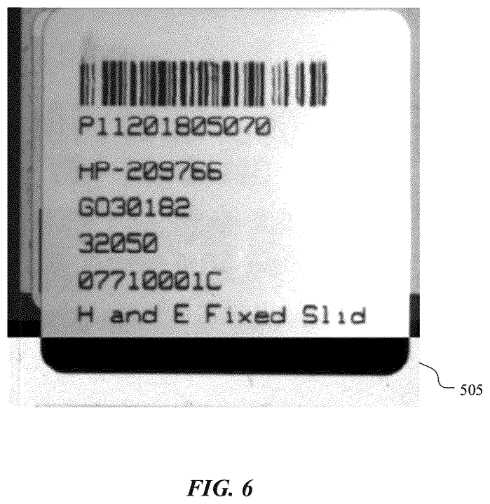

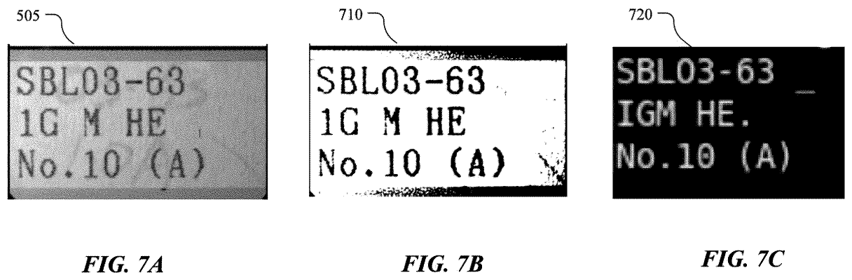

FIG. 6 illustrates an example label image 505. Once the label image 505 is available, the module for automatic label detection and recognition (AutoLDR) 230 may understand the type of metadata present either textual or digital encoded within the label image 505 and extract it with the necessary tools available. In particular embodiments, the module for automatic label detection and recognition (AutoLDR) 230 may first perform image preprocessing 510 on the label image 505. The module for automatic label detection and recognition (AutoLDR) 230 may detect an error of an orientation of the label 215 in the label image 505. The module for automatic label detection and recognition (AutoLDR) 230 may then perform orientation correction 515 by fixing the error by rotating the label image 505 based on a correct orientation of the label 215 for easier and noise-free text extraction. Based on the correction, the module for automatic label detection and recognition (AutoLDR) 230 may perform OCR text extraction 520. FIGS. 7A-7C illustrate an example process for generating an example label 215. FIG. 7A illustrates an example label image 505. FIG. 7B illustrates an example processed and oriented label image 710. FIG. 7C illustrates example extracted text 720.

The extracted text 720 may go through text postprocessing 525. On the other hand, the preprocessed label image from image preprocessing 510 may go through digital code detection 530. The detected digital code may be sent to a decoder 535. In particular embodiments, the module for automatic label detection and recognition (AutoLDR) 230 may perform metadata structuring 540 based on the processed text from text postprocessing 525 and the decoded information from the decoder 535. The module for automatic label detection and recognition (AutoLDR) 230 may then generate label textual metadata 134 and label encoded metadata 132 based on the structured metadata. The label textual metadata 134 and label encoded metadata 132 may be included in the results together with the reports 152.

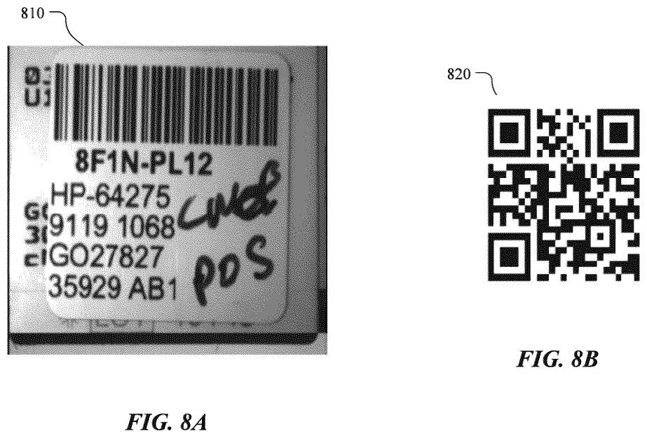

In particular embodiments, the label encoded metadata 132 may comprise the digital encoded metadata on the label 215 in various formats like barcodes, QR codes, etc. As an example and not by way of limitation, the data quality control system may deal with the following types of encoding formats, i.e., barcodes, QR codes, and data matrix, which are widely used and seen. FIGS. 8A-8B illustrate an example digital encoded label. FIG. 8A illustrates an example barcode 810 as a digital encoded label. FIG. 8B illustrates an example QR code 820 as a digital encoded label. It may be possible that sometimes there is no digital encoded metadata on the label 215. The data quality control system may use open source libraries, e.g., ZBar and ZXing, for detection and decoding of encoded metadata. ZBar may enable the data quality control system to detect the presence of barcodes and QR codes and identify the region of interest within the label 215 where these lie, upon which the data quality control system may decode information and identify the data fields extracted and format them into a tabular structured metadata file. ZBar may perform this with a throughput of decoding over 3000 labels per minute taking a little over 20 milliseconds per label. Similarly, ZXing may enable the data quality control system to decode information from data matrices with throughput of over 300 labels per minutes taking 200 milliseconds per label.

The label encoded metadata 132 may be reliable and highly capable. Since they come with their encoding standards and are machine-generated, it may be easy to decode the information. As an example and not by way of limitation, the label encoded metadata 132 may comprise one or more of a filename, a study identifier, a block identifier, or a database identifier. In particular embodiments, a block identifier may be the identifier assigned to the histology formalin-fixed paraffin-embedded (FFPE) embedded tissue blocks, which may be found on the slide as the last immediate accession identifier. Although the label encoded metadata 132 are capable of encoding large volumes of metadata, they may be underutilized and provide us with little information.

In particular embodiments, label textual metadata 134 may comprise the printed text on the label 215 or sometimes human hand-written text. The label textual metadata 134 may comprise a lot of information compared to that of label encoded metadata 132. Once a label 215 is extracted from the thumbnail image 205 it may go through a series of pre-processing steps so that the text may be highlighted, and the background noise may be removed. These pre-processing steps may comprise image blurring, illumination correction, and thresholding. Image blurring may be done to smoothen the image and close pixel-level gaps or breaks in the text. During illumination correction, the illumination intensity of the entire label may be uniformized because use of lack of proper and efficient light source the edge of the image may tend to be the darker side generating a lot of noise. Thresholding may remove the background noise and highlight the regions with text.

Once the label image 505 is pre-processed, the data quality control system may detect text, understand the orientation of the label 215, and correct it. If the text is upside down, the data quality control system may rotate it by 180 degrees. After that, the image may be fed into an open source OCR (optical character recognition) system library to recognize the characters and returns them a list of word strings. As an example and not by way of limitation, the library may be Tesseract which uses a pre-trained long short-term memory (LSTM) engine. The generated strings may have some noise like points, hyphens, or underscores, etc. attached to them, which may be removed using regular expression by simply stating the type of text pattern to be taken into account.

The extracted text may need to be assigned into their respective metadata fields. In particular embodiments, the data quality control system may format, based on a template-based pattern matching, the text to into one or more metadata fields in a tabular structure. Each vendor may have their own label structures and fields, so the data quality control system may use a template for each vendor to formulate the extracted text into tabular metadata fields and structures. In other words, the template may be determined based on the vendor metadata 122. This may make the system rely upon human feedback loop to provide the template as the label structure is not yet standardized across the board. With a standardized label structure, human intervention may be unnecessary with the system processes.

The label images 505 and metadata files in various formats like .csv and .xlsx may be stored and managed properly in their respective directories all throughout the process, providing users with easy access to source files. The generated metadata may be compared against the vendor provided metadata, checking for any possible data entry errors or missing data values. This is referred as metadata cross-validation 142, where the data quality control system may identify all the mismatched rows and generate reports 152 according to the standard.

In source file/technical metadata 138, various bio-formats may come with various types and fields of metadata embedded into the file properties. These properties may be extracted from the native slide file and may be formulated into tabular structures. Vendors may provide minimal data fields, giving the data quality control system the way to extract a wide variety of data fields from the file properties.

In particular embodiments, vendor metadata 122 may comprise some basic information like scan magnification, scan date, device type, etc. and more information relevant to the file like the image width and image height at various scan magnification levels. Using an open source tool such as OpenSlide, the data quality control system may extract rich volumes of such technical metadata.

In particular embodiments, the data quality control system may understand the type of staining used for a given slide through the module for classification of hematoxylin and eosin stained slides (CHESS) 225. Accordingly, the image content metadata 136 may comprise a type of staining used for the slide file 124. Learning this may be crucial for validation and moving data to other upstream processes. Currently, conventional work to address this quality control process may be a time-consuming human manual verification process, where an individual needs to go through each and every slide 124 in a viewer and confirm it and at the end no reusable report is generated for easy review.

When the image content metadata 136 comprises a type of staining used for the slide file, the detailed steps for determining the type of staining may be as follows. For each of the plurality of slide files, the module for classification of hematoxylin and eosin stained slides (CHESS) 225 may extract the thumbnail image 205 of the slide file 124. The thumbnail image 205 may comprise an assay associated with the corresponding slide file 124. The module for classification of hematoxylin and eosin stained slides (CHESS) 225 may then identify boundaries of the assay within the thumbnail image 205. The module for classification of hematoxylin and eosin stained slides (CHESS) 225 may then generate an assay image 210 by cropping out the assay based on the boundaries of the assay. The module for classification of hematoxylin and eosin stained slides (CHESS) 225 may further determine, based on the assay image 210, the type of staining. In particular embodiments, the determining may be further based on one or more of an amount of chemical used for staining or the one or more machine-learning models.

In particular embodiments, the module for classification of hematoxylin and eosin stained slides (CHESS) 225 may classify slides as hematoxylin and eosin (H&E) stained slides or non-H&E stained slides. The non-H&E class may comprise a wide variety of stain types like CD3, CD8, PTEN, fluorescence, etc. Through the module for classification of hematoxylin and eosin stained slides (CHESS) 225, the data quality control system may generate a new type of synthetic metadata called image content metadata 136, where the data quality control system may assess the image content to make a decision and formulate the metrics. For this purpose, the embodiments disclosed herein focus on the assay or tissue region of the thumbnail image 205. In particular embodiments, the data quality control system may extract the assay region by removing the label region from the thumbnail image 205.

In particular embodiments, there may be two approaches to implement the module for classification of hematoxylin and eosin stained slides (CHESS) 225. The first one may be a static approach where the data quality control system may perform color space binning by establishing an optimal range of values in the HSV color space that fall under the H&E stains. To further extend this and to understand possible anomalies, the data quality control system may use a scoring metric that is based on the amount of H&E stained assay content on the slide. This may be important because the data quality control system sometimes encounters slides 124 with missing assay, empty slides or even slides with minimal amounts of assay typically not usable for image analysis. As an example and not by way of limitation, such slides 124 may have their scores less than 5, 1, or even less than 1 indicating that there is a possibility of an anomaly. In particular embodiments, the system threshold may be set at 80. If the score is higher than 80, the slide may be classified as H&E stained or else as non-H&E stained.

The other approach to implement the module for classification of hematoxylin and eosin stained slides (CHESS) 225 may be based on a deep learning approach. As an example and not by way of limitation, for the deep-learning approach, the data quality control system may use a pre-trained ResNet18 architecture model that is trained on 250 slides of each H&E and non-H&E class. The pre-trained ResNet18 architecture model may be able to achieve an accuracy of over 99% by minimizing the losses to less than 1%. The embodiments disclosed herein gathered over 257 slides of H&E class and 304 slides of non-H&E class. These slides may comprise various types of tissues and the data quality control system has proven to be tissue independent.

When the image content metadata 136 comprises one or more types of tissue of the slide file 124, the detailed steps of determining the types of the tissue of the slide file 124 may be as follows. For each of the plurality of slide files 124, the module for tissue identification (TiD) 220 may extract the thumbnail image 205 of the slide file 124. The thumbnail image 205 may comprise an assay associated with the corresponding slide file 124. The module for tissue identification (TiD) 220 may then identify boundaries of the assay within the thumbnail image 205. The module for tissue identification (TiD) 220 may then generate an assay image 210 by cropping out the assay based on the boundaries of the assay. The module for tissue identification (TiD) 220 may then detect one or more assay pieces within the assay image 210. The module for tissue identification (TiD) 220 may then segment the one or more assay pieces. The module for tissue identification (TiD) 220 may further determine, based on the segmented one or more assay pieces, the one or more types of tissue by the one or more machine-learning models.

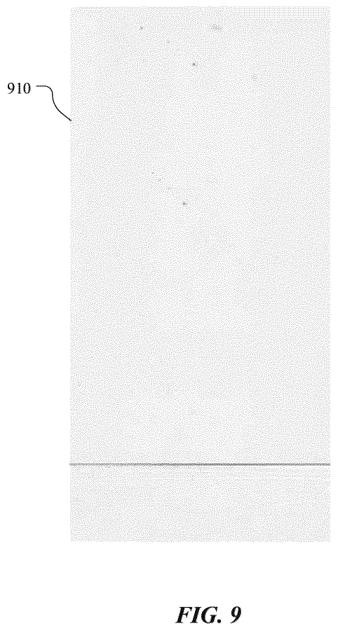

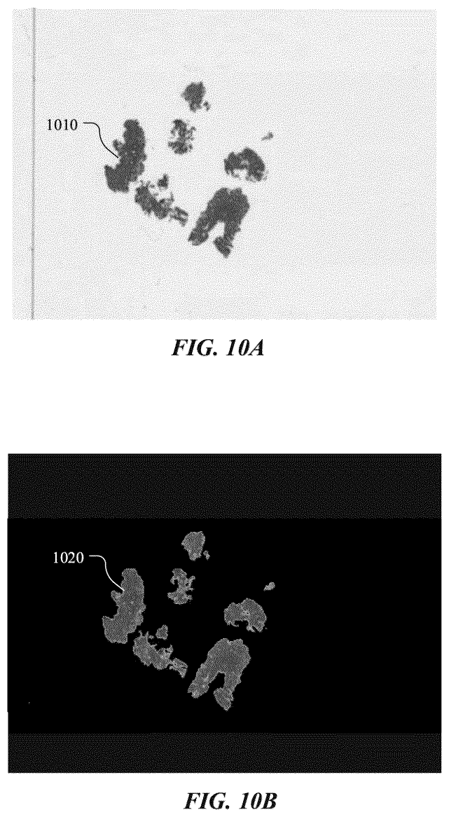

In particular embodiments, the module for tissue identification (TiD) 220 may address the often missing or unknown data field and the anatomical tissue type which is very important. FIG. 9 illustrates an example empty slide. The module for tissue identification (TiD) 220 may determine that the slide 910 in FIG. 9 is empty. Through the module for tissue identification (TiD) 220, the data quality control system may identify the type of tissue in the slide 124. Accordingly, the image content metadata 136 may comprise a type of tissue of the slide file 124. As an example and not by way of limitation, the data quality control system may identify slides 124 with single anatomical tissue. In a real-world scenario, multiple pieces of different types of tissues may be present on the same slide 124. The module for tissue identification (TiD) 220 may address this by identifying the number of assay pieces on the slides 124, then segmenting each piece separately, and further passing it to the system to get the tissue type of each individual piece and generate reports 152. FIGS. 10A-10B illustrate an example identification of a tissue. FIG. 10A illustrates an example tissue region 1010. In particular embodiments, the module for tissue identification (TiD) 220 may first identify the tissue region. The module for tissue identification (TiD) 220 may then segment the tissue region. FIG. 10B illustrates an example segmented tissue region 1020. The module for tissue identification (TiD) 220 may further identify the tissue based on the segmented the tissue region.

As an example and not by way of limitation, the module for tissue identification (TiD) 220 may identify these types of tissues, e.g., bladder, breast and colon. The module for tissue identification (TiD) 220 may also identify the more anatomical tissue locations. In particular embodiments, the module for tissue identification (TiD) 220 may be based on a deep-learning approach. As an example and not by way of limitation, for the deep-learning approach, the data quality control system may use a pretrained ResNet152 architecture model that is trained over 240 slides of each type of tissue and the assay regions extracted from the thumbnail images 205. The embodiments disclosed herein gathered 641 slides for bladder, 330 slides for breast and 240 for colon. The slides collected have various stain types and the module for tissue identification (TiD) 220 was able to perform identification independent of the stain type and achieved an accuracy of over 96.5% while reducing the losses to less than 3%.

In particular embodiments, the data quality control system may provide an end user interface 154. Through this easy-to-use interface 154, the users (e.g., data consumers) may review various types of tabular metadata structures and the results generated by various modules with quick preview abilities to view both the assay and label regions of the thumbnail image 205 extracted. In particular embodiments, user profiles and access control tools may be implemented to support the various needs of end users. An integrated process request tool may be developed to enable users to request reports as per their needs.

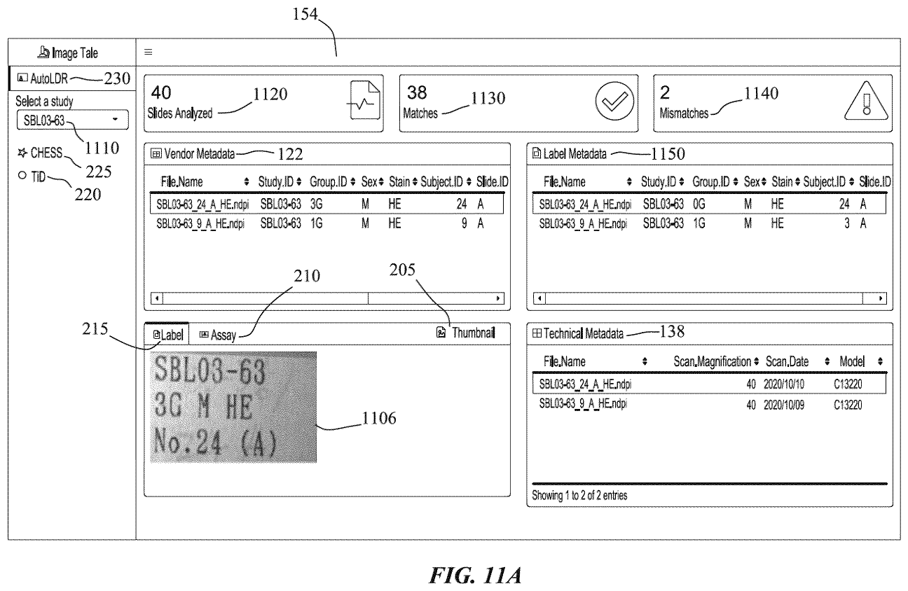

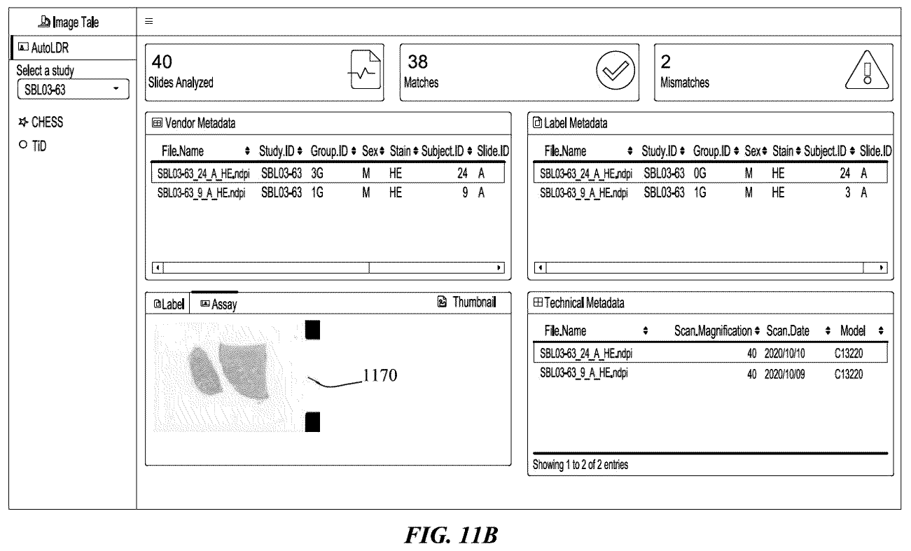

In particular embodiments, the user interface 154 may follow an easy-to-navigate dashboard structure, with key metrics defined and showcased on the top, for which the embodiments disclosed herein may refer the user interface as dashboard interface 154. All the key information may be color-coded, with headers placed in their respective container like cells. Most of the metadata information may be represented in a tabularized format making it easy to interpret and use for the end user. FIGS. 11A-11B illustrates an example dashboard interface 154. On the left, the dashboard interface 154 may comprise three options for the three modules, i.e., AutoLDR 230, CHESS 225, and TiD 220. In particular embodiments, the report 152 may comprise content specific to each module. The user interface 154 may be operable for the user to view the content specific to each module separately. In other words, a user may select each of them to view the results from that module. As an example and not by way of limitation, FIGS. 11A-11B may show that the user is currently viewing the results from AutoLDR 230. The user may select a study (e.g., SBL03-63) from a drop-down menu 1110. On the right, the dashboard interface 154 may show slides analyzed 1120, matches 1130, mismatches 1140, vendor metadata 122, label metadata 1150 comprising both label encoded metadata 132 and label textual metadata 134, thumbnail 205 comprising label 215 and assay 210, source file/technical metadata 138. As aforementioned, each of the vendor metadata 122, label metadata 1150, and technical metadata 138 may be based on a tabular structure comprising one or more metadata fields. As a result, the matches 1130 and mismatches 1140 may be determined based on comparisons between the metadata fields of the vendor metadata 122 and the corresponding metadata fields of the label metadata 1150 and technical metadata 138, respectively. As displayed in FIGS. 11A-11B, there may be 40 slides analyzed, among which there are 38 matches and 2 mismatches. The two mismatches may be listed in the tables of vendor metadata 122, label metadata 1150, and technical metadata 138. In particular embodiments, the user interface 154 may display the vendor metadata 122, label metadata 1150, and technical metadata 138 in the respective tabular structure. In FIGS. 11A-11B, the tables in vendor metadata 122 each has a horizontal scroll bar and it may have more fields than the table in label metadata 1150, which may be not shown in FIGS. 11A-11B. FIG. 11A illustrates an example dashboard interface 154 with label view. As illustrated in FIG. 11A, the user may have selected the first file in vendor metadata 122 with a file name “SBL03-63_24_A_HE.ndpi”. In the section of thumbnail 205, the user may have selected label 215. Accordingly, there may be a label 1160 shown corresponding to the file “SBL03-63_24_A_HE.ndpi”. FIG. 11B illustrates an example user interface with assay view. As illustrated in FIG. 11B, in the section of thumbnail 205, the user may have selected assay 210. Accordingly, there may be an assay image 1170 shown corresponding to the file “SBL03-63_24_A_HE.ndpi”.

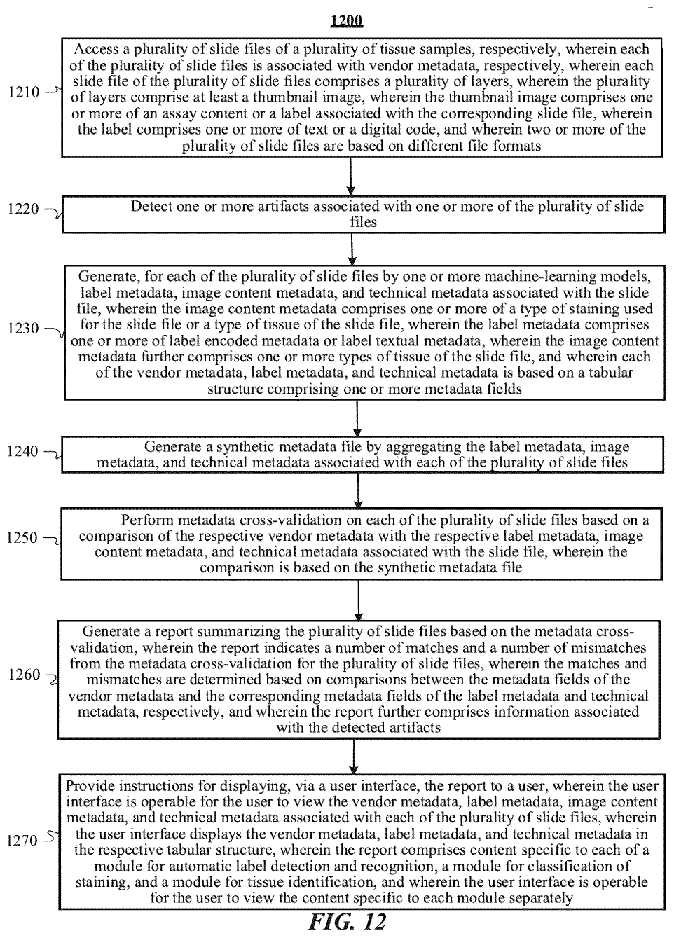

FIG. 12 illustrates an example method 1200 for data quality control and data integration. The method may begin at step 1210, where the data quality control system may access a plurality of slide files of a plurality of tissue samples, respectively, wherein each of the plurality of slide files is associated with vendor metadata, respectively, wherein each slide file of the plurality of slide files comprises a plurality of layers, wherein the plurality of layers comprise at least a thumbnail image, wherein the thumbnail image comprises one or more of an assay content or a label associated with the corresponding slide file, wherein the label comprises one or more of text or a digital code, and wherein two or more of the plurality of slide files are based on different file formats. At step 1220, the data quality control system may detect one or more artifacts associated with one or more of the plurality of slide files. At step 1230, the data quality control system may generate, for each of the plurality of slide files by one or more machine-learning models, label metadata, image content metadata, and technical metadata associated with the slide file, wherein the image content metadata comprises one or more of a type of staining used for the slide file or a type of tissue of the slide file, wherein the label metadata comprises one or more of label encoded metadata or label textual metadata, wherein the image content metadata further comprises one or more types of tissue of the slide file, and wherein each of the vendor metadata, label metadata, and technical metadata is based on a tabular structure comprising one or more metadata fields. At step 1240, the data quality control system may generate a synthetic metadata file by aggregating the label metadata, image metadata, and technical metadata associated with each of the plurality of slide files. At step 1250, the data quality control system may perform metadata cross-validation on each of the plurality of slide files based on a comparison of the respective vendor metadata with the respective label metadata, image content metadata, and technical metadata associated with the slide file, wherein the comparison is based on the synthetic metadata file. At step 1260, the data quality control system may generate a report summarizing the plurality of slide files based on the metadata cross-validation, wherein the report indicates a number of matches and a number of mismatches from the metadata cross-validation for the plurality of slide files, wherein the matches and mismatches are determined based on comparisons between the metadata fields of the vendor metadata and the corresponding metadata fields of the label metadata and technical metadata, respectively, and wherein the report further comprises information associated with the detected artifacts. At step 1270, the data quality control system may provide instructions for displaying, via a user interface, the report to a user, wherein the user interface is operable for the user to view the vendor metadata, label metadata, image content metadata, and technical metadata associated with each of the plurality of slide files, wherein the user interface displays the vendor metadata, label metadata, and technical metadata in the respective tabular structure, wherein the report comprises content specific to each of a module for automatic label detection and recognition, a module for classification of staining, and a module for tissue identification, and wherein the user interface is operable for the user to view the content specific to each module separately. Particular embodiments may repeat one or more steps of the method of FIG. 12, where appropriate. Although this disclosure describes and illustrates particular steps of the method of FIG. 12 as occurring in a particular order, this disclosure contemplates any suitable steps of the method of FIG. 12 occurring in any suitable order. Moreover, although this disclosure describes and illustrates an example method for data quality control and data integration, including the particular steps of the method of FIG. 12, this disclosure contemplates any suitable method for data quality control and data integration, including any suitable steps, which may include all, some, or none of the steps of the method of FIG. 12, where appropriate. Furthermore, although this disclosure describes and illustrates particular components, devices, or systems carrying out particular steps of the method of FIG. 12, this disclosure contemplates any suitable combination of any suitable components, devices, or systems carrying out any suitable steps of the method of FIG. 12.

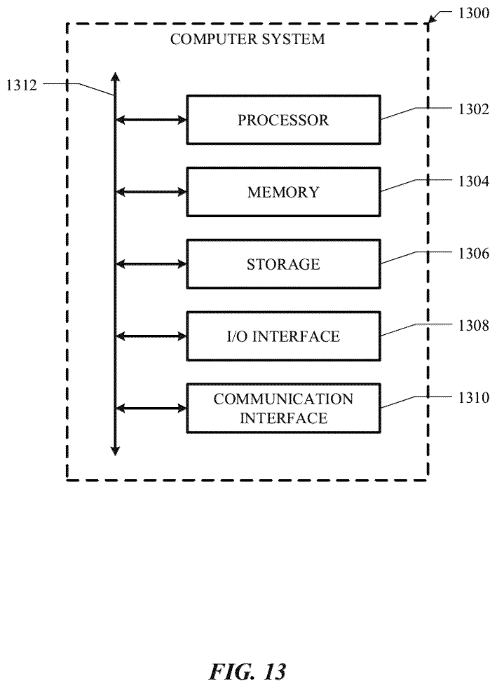

FIG. 13 illustrates an example computer system 1300. In particular embodiments, one or more computer systems 1300 perform one or more steps of one or more methods described or illustrated herein. In particular embodiments, one or more computer systems 1300 provide functionality described or illustrated herein. In particular embodiments, software running on one or more computer systems 1300 performs one or more steps of one or more methods described or illustrated herein or provides functionality described or illustrated herein. Particular embodiments include one or more portions of one or more computer systems 1300. Herein, reference to a computer system may encompass a computing device, and vice versa, where appropriate. Moreover, reference to a computer system may encompass one or more computer systems, where appropriate.

This disclosure contemplates any suitable number of computer systems 1300. This disclosure contemplates computer system 1300 taking any suitable physical form. As example and not by way of limitation, computer system 1300 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1300 may include one or more computer systems 1300; be unitary or distributed; span multiple locations; span multiple machines; span multiple data centers; or reside in a cloud, which may include one or more cloud components in one or more networks. Where appropriate, one or more computer systems 1300 may perform without substantial spatial or temporal limitation one or more steps of one or more methods described or illustrated herein. As an example and not by way of limitation, one or more computer systems 1300 may perform in real time or in batch mode one or more steps of one or more methods described or illustrated herein. One or more computer systems 1300 may perform at different times or at different locations one or more steps of one or more methods described or illustrated herein, where appropriate.

In particular embodiments, computer system 1300 includes a processor 1302, memory 1304, storage 1306, an input/output (I/O) interface 1308, a communication interface 1310, and a bus 1312. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1302 includes hardware for executing instructions, such as those making up a computer program. As an example and not by way of limitation, to execute instructions, processor 1302 may retrieve (or fetch) the instructions from an internal register, an internal cache, memory 1304, or storage 1306; decode and execute them; and then write one or more results to an internal register, an internal cache, memory 1304, or storage 1306. In particular embodiments, processor 1302 may include one or more internal caches for data, instructions, or addresses. This disclosure contemplates processor 1302 including any suitable number of any suitable internal caches, where appropriate. As an example and not by way of limitation, processor 1302 may include one or more instruction caches, one or more data caches, and one or more translation lookaside buffers (TLBs). Instructions in the instruction caches may be copies of instructions in memory 1304 or storage 1306, and the instruction caches may speed up retrieval of those instructions by processor 1302. Data in the data caches may be copies of data in memory 1304 or storage 1306 for instructions executing at processor 1302 to operate on; the results of previous instructions executed at processor 1302 for access by subsequent instructions executing at processor 1302 or for writing to memory 1304 or storage 1306; or other suitable data. The data caches may speed up read or write operations by processor 1302. The TLBs may speed up virtual-address translation for processor 1302. In particular embodiments, processor 1302 may include one or more internal registers for data, instructions, or addresses. This disclosure contemplates processor 1302 including any suitable number of any suitable internal registers, where appropriate. Where appropriate, processor 1302 may include one or more arithmetic logic units (ALUs); be a multi-core processor; or include one or more processors 1302. Although this disclosure describes and illustrates a particular processor, this disclosure contemplates any suitable processor.

In particular embodiments, memory 1304 includes main memory for storing instructions for processor 1302 to execute or data for processor 1302 to operate on. As an example and not by way of limitation, computer system 1300 may load instructions from storage 1306 or another source (such as, for example, another computer system 1300) to memory 1304. Processor 1302 may then load the instructions from memory 1304 to an internal register or internal cache. To execute the instructions, processor 1302 may retrieve the instructions from the internal register or internal cache and decode them. During or after execution of the instructions, processor 1302 may write one or more results (which may be intermediate or final results) to the internal register or internal cache. Processor 1302 may then write one or more of those results to memory 1304. In particular embodiments, processor 1302 executes only instructions in one or more internal registers or internal caches or in memory 1304 (as opposed to storage 1306 or elsewhere) and operates only on data in one or more internal registers or internal caches or in memory 1304 (as opposed to storage 1306 or elsewhere). One or more memory buses (which may each include an address bus and a data bus) may couple processor 1302 to memory 1304. Bus 1312 may include one or more memory buses, as described below. In particular embodiments, one or more memory management units (MMUs) reside between processor 1302 and memory 1304 and facilitate accesses to memory 1304 requested by processor 1302. In particular embodiments, memory 1304 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1304 may include one or more memories 1304, where appropriate. Although this disclosure describes and illustrates particular memory, this disclosure contemplates any suitable memory.

In particular embodiments, storage 1306 includes mass storage for data or instructions. As an example and not by way of limitation, storage 1306 may include a hard disk drive (HDD), a floppy disk drive, flash memory, an optical disc, a magneto-optical disc, magnetic tape, or a Universal Serial Bus (USB) drive or a combination of two or more of these. Storage 1306 may include removable or non-removable (or fixed) media, where appropriate. Storage 1306 may be internal or external to computer system 1300, where appropriate. In particular embodiments, storage 1306 is non-volatile, solid-state memory. In particular embodiments, storage 1306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1306 taking any suitable physical form. Storage 1306 may include one or more storage control units facilitating communication between processor 1302 and storage 1306, where appropriate. Where appropriate, storage 1306 may include one or more storages 1306. Although this disclosure describes and illustrates particular storage, this disclosure contemplates any suitable storage.

In particular embodiments, I/O interface 1308 includes hardware, software, or both, providing one or more interfaces for communication between computer system 1300 and one or more I/O devices. Computer system 1300 may include one or more of these I/O devices, where appropriate. One or more of these I/O devices may enable communication between a person and computer system 1300. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1308 for them. Where appropriate, I/O interface 1308 may include one or more device or software drivers enabling processor 1302 to drive one or more of these I/O devices. I/O interface 1308 may include one or more I/O interfaces 1308, where appropriate. Although this disclosure describes and illustrates a particular I/O interface, this disclosure contemplates any suitable I/O interface.

In particular embodiments, communication interface 1310 includes hardware, software, or both providing one or more interfaces for communication (such as, for example, packet-based communication) between computer system 1300 and one or more other computer systems 1300 or one or more networks. As an example and not by way of limitation, communication interface 1310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1310 for it. As an example and not by way of limitation, computer system 1300 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1300 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1300 may include any suitable communication interface 1310 for any of these networks, where appropriate. Communication interface 1310 may include one or more communication interfaces 1310, where appropriate. Although this disclosure describes and illustrates a particular communication interface, this disclosure contemplates any suitable communication interface.

In particular embodiments, bus 1312 includes hardware, software, or both coupling components of computer system 1300 to each other. As an example and not by way of limitation, bus 1312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1312 may include one or more buses 1312, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

### EMBODIMENTS

Among the provided embodiments are:

1. A method comprising, by a data quality control system:
accessing a plurality of slide files of a plurality of tissue samples, respectively, wherein each of the plurality of slide files is associated with vendor metadata, respectively;
generating, for each of the plurality of slide files by one or more machine-learning models, label metadata, image content metadata, and technical metadata associated with the slide file;
performing metadata cross-validation on each of the plurality of slide files based on a comparison of the respective vendor metadata with the respective label metadata, image content metadata, and technical metadata associated with the slide file;
generating a report summarizing the plurality of slide files based on the metadata cross-validation, wherein the report indicates a number of matches and a number of mismatches from the metadata cross-validation for the plurality of slide files; and
providing instructions for displaying, via a user interface, the report to a user, wherein the user interface is operable for the user to view the vendor metadata, label metadata, image content metadata, and technical metadata associated with each of the plurality of slide files.

2. The method of Embodiment 1, wherein the image content metadata comprises one or more of a type of staining used for the slide file or a type of tissue of the slide file.

3. The method of any of Embodiments 1-2, wherein the label metadata comprises one or more of label encoded metadata or label textual metadata.

4. The method of any of Embodiments 1-3, wherein each slide file of the plurality of slide files comprises a plurality of layers, wherein the plurality of layers comprise at least a thumbnail image, and wherein the thumbnail image comprises one or more of an assay content or a label associated with the corresponding slide file, wherein the label comprises one or more of text or a digital code.

5. The method of any of Embodiments 1-4, wherein the label metadata comprises label encoded metadata, wherein the method further comprises:
for each of the plurality of slide files:
   extracting the thumbnail image of the slide file, wherein the thumbnail image comprises a label associated with the corresponding slide file;
   identifying boundaries of the label within the thumbnail image;
   generating a label image by cropping out the label based on the boundaries of the label;
   detecting a presence of a digital code in the label image; and
   generating the label encoded metadata based on decoding the digital code, wherein the label encoded metadata comprises one or more of a filename, a study identifier, a block identifier, or a database identifier.

6. The method of any of Embodiments 1-5, further comprising:
detecting an error of an orientation of the label in the label image; and
fixing the error by rotating the label image based on a correct orientation of the label.

7. The method of any of Embodiments 1-6, wherein the label metadata comprises label textual metadata, wherein the method further comprises:
for each of the plurality of slide files:
   extracting the thumbnail image of the slide file, wherein the thumbnail image comprises a label associated with the corresponding slide file;
   identifying boundaries of the label within the thumbnail image;
   generating a label image by cropping out the label based on the boundaries of the label;
   preprocessing the label image, wherein the preprocessing comprises one or more of image blurring, illumination correction, or thresholding;
   detecting text in the preprocessed label image; and
   generating the label textual metadata based on optical character recognition on the detected text.

8. The method of any of Embodiments 1-7, further comprising:
formatting, based on a template-based pattern matching, the text to into one or more metadata fields in a tabular structure, wherein the template is determined based on the vendor metadata.

9. The method of any of Embodiments 1-8, wherein the image content metadata comprises a type of staining used for the slide file, wherein the method further comprises:
for each of the plurality of slide files:
   extracting the thumbnail image of the slide file, wherein the thumbnail image comprises an assay associated with the corresponding slide file;
   identifying boundaries of the assay within the thumbnail image;
   generating an assay image by cropping out the assay based on the boundaries of the assay; and
   determining, based on the assay image, the type of staining, wherein the determining is further based on one or more of an amount of chemical used for staining or the one or more machine-learning models.

10. The method of any of Embodiments 1-9, wherein the image content metadata comprises one or more types of tissue of the slide file, wherein the method further comprises, for each of the plurality of slide files:
extracting the thumbnail image of the slide file, wherein the thumbnail image comprises an assay associated with the corresponding slide file;
identifying boundaries of the assay within the thumbnail image;
generating an assay image by cropping out the assay based on the boundaries of the assay;
detecting one or more assay pieces within the assay image;
segmenting the one or more assay pieces; and
determining, based on the segmented one or more assay pieces, the one or more types of tissue by the one or more machine-learning models.

11. The method of any of Embodiments 1-10, wherein two or more of the plurality of slide files are based on different file formats.

12. The method of any of Embodiments 1-11, further comprising:
generating a synthetic metadata file by aggregating the label metadata, image metadata, and technical metadata associated with each of the plurality of slide files, wherein the comparison is based on the synthetic metadata file.

13. The method of any of Embodiments 1-12, wherein the data quality control system is based on a plurality of modules comprising a module for automatic label detection and recognition, a module for classification of staining, and a module for tissue identification, wherein the report comprises content specific to each module, and wherein the user interface is operable for the user to view the content specific to each module separately.

14. The method of any of Embodiments 1-13, wherein each of the vendor metadata, label metadata, and technical metadata is based on a tabular structure comprising one or more metadata fields, and wherein the matches and mismatches are determined based on comparisons between the metadata fields of the vendor metadata and the corresponding metadata fields of the label metadata and technical metadata, respectively.

15. The method of any of Embodiments 1-14, wherein the user interface displays the vendor metadata, label metadata, and technical metadata in the respective tabular structure.

16. The method of any of Embodiments 1-15, further comprising:
detecting one or more artifacts associated with one or more of the plurality of slide files, wherein the report further comprises information associated with the detected artifacts.

17. One or more computer-readable non-transitory storage media embodying software that is operable when executed by one or more processors to perform the steps of any of Embodiments 1 to 16.

18. A system comprising: one or more processors; and a non-transitory memory coupled to the processors comprising instructions executable by the one or more processors, the one or more processors operable when executing the instructions to perform the steps of any of Embodiments 1 to 16.

What is claimed is:

1. A method comprising, by a data quality control system:
accessing a plurality of slide files of a plurality of tissue samples, respectively, wherein each of the plurality of slide files is associated with vendor metadata, respectively;
generating, for each of the plurality of slide files by one or more machine-learning models, label metadata, image content metadata, and technical metadata associated with the slide file;
performing metadata cross-validation on each of the plurality of slide files based on a comparison of the respective vendor metadata with the respective label metadata, image content metadata, and technical metadata associated with the slide file;
generating a report summarizing the plurality of slide files based on the metadata cross-validation, wherein the report indicates a number of matches and a number of mismatches from the metadata cross-validation for the plurality of slide files; and
providing instructions for displaying, via a user interface, the report to a user, wherein the user interface is operable for the user to view the vendor metadata, label metadata, image content metadata, and technical metadata associated with each of the plurality of slide files.

2. The method of claim 1, wherein the image content metadata comprises one or more of a type of staining used for the slide file or a type of tissue of the slide file.

3. The method of claim 1, wherein the label metadata comprises one or more of label encoded metadata or label textual metadata.

4. The method of claim 1, wherein each slide file of the plurality of slide files comprises a plurality of layers, wherein the plurality of layers comprise at least a thumbnail image, and wherein the thumbnail image comprises one or more of an assay content or a label associated with the corresponding slide file, wherein the label comprises one or more of text or a digital code.

5. The method of claim 4, wherein the label metadata comprises label encoded metadata, wherein the method further comprises:
for each of the plurality of slide files:
extracting the thumbnail image of the slide file, wherein the thumbnail image comprises a label associated with the corresponding slide file;
identifying boundaries of the label within the thumbnail image;
generating a label image by cropping out the label based on the boundaries of the label;
detecting a presence of a digital code in the label image; and
generating the label encoded metadata based on decoding the digital code, wherein the label encoded metadata comprises one or more of a filename, a study identifier, a block identifier, or a database identifier.

6. The method of claim 5, further comprising:
detecting an error of an orientation of the label in the label image; and
fixing the error by rotating the label image based on a correct orientation of the label.

7. The method of claim 4, wherein the label metadata comprises label textual metadata, wherein the method further comprises:
for each of the plurality of slide files:
extracting the thumbnail image of the slide file, wherein the thumbnail image comprises a label associated with the corresponding slide file;
identifying boundaries of the label within the thumbnail image;
generating a label image by cropping out the label based on the boundaries of the label;
preprocessing the label image, wherein the preprocessing comprises one or more of image blurring, illumination correction, or thresholding;
detecting text in the preprocessed label image; and
generating the label textual metadata based on optical character recognition on the detected text.

8. The method of claim 7, further comprising:
formatting, based on a template-based pattern matching, the text to into one or more metadata fields in a tabular structure, wherein the template is determined based on the vendor metadata.

9. The method of claim 4, wherein the image content metadata comprises a type of staining used for the slide file, wherein the method further comprises:
for each of the plurality of slide files:
extracting the thumbnail image of the slide file, wherein the thumbnail image comprises an assay associated with the corresponding slide file;
identifying boundaries of the assay within the thumbnail image;
generating an assay image by cropping out the assay based on the boundaries of the assay; and
determining, based on the assay image, the type of staining, wherein the determining is further based on one or more of an amount of chemical used for staining or the one or more machine-learning models.

10. The method of claim 1, wherein the image content metadata comprises one or more types of tissue of the slide file, wherein the method further comprises, for each of the plurality of slide files:
extracting the thumbnail image of the slide file, wherein the thumbnail image comprises an assay associated with the corresponding slide file;
identifying boundaries of the assay within the thumbnail image;
generating an assay image by cropping out the assay based on the boundaries of the assay;
detecting one or more assay pieces within the assay image;
segmenting the one or more assay pieces; and
determining, based on the segmented one or more assay pieces, the one or more types of tissue by the one or more machine-learning models.

11. The method of claim 1, wherein two or more of the plurality of slide files are based on different file formats.

12. The method of claim 1, further comprising:
generating a synthetic metadata file by aggregating the label metadata, image metadata, and technical metadata associated with each of the plurality of slide files, wherein the comparison is based on the synthetic metadata file.

13. The method of claim 1, wherein the data quality control system is based on a plurality of modules comprising a module for automatic label detection and recognition, a module for classification of staining, and a module for tissue identification, wherein the report comprises content specific to each module, and wherein the user interface is operable for the user to view the content specific to each module separately.

14. The method of claim 1, wherein each of the vendor metadata, label metadata, and technical metadata is based on a tabular structure comprising one or more metadata fields, and wherein the matches and mismatches are determined based on comparisons between the metadata fields of the vendor metadata and the corresponding metadata fields of the label metadata and technical metadata, respectively.

15. The method of claim 14, wherein the user interface displays the vendor metadata, label metadata, and technical metadata in the respective tabular structure.

16. The method of claim 1, further comprising:
detecting one or more artifacts associated with one or more of the plurality of slide files, wherein the report further comprises information associated with the detected artifacts.

17. One or more computer-readable non-transitory storage media embodying software that is operable when executed to:
access a plurality of slide files of a plurality of tissue samples, respectively, wherein each of the plurality of slide files is associated with vendor metadata, respectively;
generate, for each of the plurality of slide files by one or more machine-learning models, label metadata, image content metadata, and technical metadata associated with the slide file;
perform metadata cross-validation on each of the plurality of slide files based on a comparison of the respective vendor metadata with the respective label metadata, image content metadata, and technical metadata associated with the slide file;
generate a report summarizing the plurality of slide files based on the metadata cross-validation, wherein the report indicates a number of matches and a number of mismatches from the metadata cross-validation for the plurality of slide files; and
provide instructions for displaying, via a user interface, the report to a user, wherein the user interface is operable for the user to view the vendor metadata, label metadata, image content metadata, and technical metadata associated with each of the plurality of slide files.

18. The media of claim 17, wherein the data quality control system is based on a plurality of modules comprising a module for automatic label detection and recognition, a module for classification of staining, and a module for tissue identification, wherein the report comprises content specific to each module, and wherein the user interface is operable for the user to view the content specific to each module separately.

19. A system comprising: one or more processors; and a non-transitory memory coupled to the processors comprising instructions executable by the processors, the processors operable when executing the instructions to:
access a plurality of slide files of a plurality of tissue samples, respectively, wherein each of the plurality of slide files is associated with vendor metadata, respectively;
generate, for each of the plurality of slide files by one or more machine-learning models, label metadata, image content metadata, and technical metadata associated with the slide file;
perform metadata cross-validation on each of the plurality of slide files based on a comparison of the respective vendor metadata with the respective label metadata, image content metadata, and technical metadata associated with the slide file;
generate a report summarizing the plurality of slide files based on the metadata cross-validation, wherein the report indicates a number of matches and a number of mismatches from the metadata cross-validation for the plurality of slide files; and
provide instructions for displaying, via a user interface, the report to a user, wherein the user interface is operable for the user to view the vendor metadata, label metadata, image content metadata, and technical metadata associated with each of the plurality of slide files.

20. The system of claim 19, wherein the data quality control system is based on a plurality of modules comprising a module for automatic label detection and recognition, a module for classification of staining, and a module for tissue identification, wherein the report comprises content specific to each module, and wherein the user interface is operable for the user to view the content specific to each module separately.
